(19) United States
(12) Patent Application Publication
(10) Pub. No.: US 2020/0129353 A1
(43) Pub. Date: Apr. 30, 2020

(54) REMOTE MANAGEMENT, CONTROL AND ALERT SYSTEM FOR MEDICAL AIR ALTERNATION MATTRESS

(71) Applicants: SEQUOIA-TWN HEALTHCARE CO., LTD., New Taipei City (TW); Russidan Holdings Pty Ltd., Kirribilli (AU)

(72) Inventor: Ming-Tso Su, New Taipei City (TW)

(21) Appl. No.: 16/171,323

(22) Filed: Oct. 25, 2018

(51) Int. Cl. A61G 7/057; G08B 21/04; A47C 27/08; A47C 31/00; A47C 27/10; A47C 20/04; A61H 9/00; A61B 5/00

(52) U.S. Cl. CPC A61G 7/05776; G08B 21/0461; A47C 27/083; A47C 31/008; A47C 27/084; A47C 27/10; A61H 2203/0456; A61H 9/0078; A61B 5/447; A61B 5/746; A61G 2203/34; A61G 2203/20; A47C 20/048

(57) ABSTRACT

A remote management, control and alert system for a medical air alternation mattress provided in the present disclosure comprises an integration and connection between internal and external devices/units. An internal pressure control device connected with at least one air inflation/deflation unit which connects with a plurality of air cushion units in an air mattress for regulated pressure of the air cushion units in the air mattress through the air inflation/deflation unit in a controlled manner; furthermore, the pressure control device is further connected with a manipulation device to operate or analyze the data processing device through the manipulation device, for controlled regulation of pressures in all parts of the air mattress. These aforementioned internal devices/units are then in turn connected to and integrated with an external managing device allowing the user to control, monitor and receive alert notifications for the entire integrated system or systems to which it is connected.

### BACKGROUND OF THE INVENTION

### Field of the Invention

The present disclosure relates to a remote management, control and alert system for a medical air alternation mattress, particularly a remote management, control and alert system employing a pressure output control device and an inflation/deflation unit to regulate pressures in an air mattress.

### Description of the Prior Art

People with serious medical conditions which restrict their ability to reposition themselves are assessed at high risk of developing pressure ulcers. This occurs because static body weight, especially over bony prominences for extended periods of time, can prevent blood from supplying tissue under these body prominences. If this body weight is not relieved, the tissue in question becomes necrotic and a pressure ulcer develops.

Modern clinical care for such patients requires the use of an air alternating mattress in which air pressure is applied to and released from a series of air cushion units under the patient. The patient is suspended by one set of cushion units whilst another is deflated away allowing reperfusion of blood flow to affected body tissue.

However, there are many occasions where an alternating air system fails to effectively treat or prevent pressure areas from occurring. These are mostly due to the following factors. Insufficient pressure being set so that the patient falls through the supporting cells and no longer receives therapy. Power or air hoses being accidentally disconnected causing the system to deflate or stop functioning. Gradual loss of effective pump function due to age or internal damage. Incorrect choice of alternating system to match patient weight or condition. System not adjusted to compensate for non standard patient weight distribution on the mattress such as amputees or those needing to be constantly raised for intubation or treatment of respiratory conditions. Accordingly, the patent applicant proposes a unique remote management, control and alert system for a medical air alternation mattress to overcome the risk of these failures occurring.

### SUMMARY OF THE INVENTION

To overcome these issues, a remote management, control and alert system for a medical air alternation mattress provided in the present disclosure relies on a unique integration between devices both internal and external to the mattress system and its control unit. A description of these internal systems includes a pressure control device which inflates/deflates an air alternating mattress through an air inflation/deflation unit. This in turn directly controls and regulates the pressures of all air cushion units in the air mattress. Moreover, this remote management, control and alert system also depends on a manipulation device to monitor and operate the pressure control device and an internal data processing device to analyze and regulate the connected air mattress so as to control the pressures throughout the air mattress effectively. The aim of these integrated and connected internal systems is to effectively balance support pressure with timed pressure release so that the patient is both comfortable and being effectively treated via pressure release over alternately deflating air cushion units.

A remote management, control and alert system for an air mattress provided in the present disclosure comprises the following internally connected and integrated systems: an air mattress with a plurality of air cushion units, each of which is connected with an air inflation/deflation unit, inflated/deflated independently and equipped with a pressure sensor unit for generation of current pressure in each air cushion unit; an air supply device connected with the air inflation/deflation unit for supply of air to all air cushion units from the air supply device through the air inflation/deflation unit; a pressure control device connected with the air inflation/deflation unit and all pressure sensor units and used to regulate actions of the air inflation/deflation unit and adjust pressures of the air cushion units for generation of pressure monitoring information based on pressures of the air cushion units and transmissions of the pressure monitoring information as well as basic information configured in the pressure control device to outside environment; a manipulation device connected with the pressure control device through which an air inflation/deflation unit connecting a pressure control unit is controlled for a regulated pressure of an air cushion unit; a data processing device connected with the pressure control device and used to receive the basic information and the pressure monitoring information for generation of pressure regulation information based on the pressure monitoring information wherein the pressure control device relies on the pressure regulation information to regulate pressures of the air inflation/deflation unit.

Preferably, the pressure control device comprises: a pressure control and transfer unit connected with a configuration unit for receiving basic information configured at the configuration unit by a user and the current pressure of an air cushion unit detected by a pressure sensor unit and transmitting this basic information to the outside environment from the pressure control and transfer unit; a pressure control unit, which is connected with the pressure control and transfer unit for receiving current pressures of the air cushion units detected by the pressure sensor units and also connected with the air inflation/deflation unit for inflating/deflating the air mattress by the air inflation/deflation unit controllably; a monitoring unit connected with the pressure control unit through which current pressures of the air cushion units are transmitted to the monitoring unit for generation of pressure monitoring information by the monitoring unit based on pressures of the air cushion units and transmission of the pressure monitoring information from the monitoring unit to the pressure control unit, the pressure control and transfer unit, and outside environment sequentially.

Preferably, the manipulation device comprises: a manipulation and transfer unit through which configured information is transmitted to the pressure control and transfer unit; a configuration unit from which configured information based on a user's configurations is generated; a control unit connected with the configuration unit for receiving the configured information and also connected with the manipulation and transfer unit for transmitting the configured information to the manipulation and transfer unit such that pressure of the air inflation/deflation unit is regulated by the pressure control device controllably according to the configured information.

Preferably, the configuration unit comprises: a plurality of function configuration parts, each of which is operated for generation of configured information; at least one status display part installed around a function configuration part for displaying current configurations of the air mattress.

Preferably, the data processing device comprises: a data processing and transfer unit connected with the pressure control and transfer unit for receiving the basic information and the pressure monitoring information; a data processing and control unit connected with the data processing and transfer unit for receiving the basic information and the pressure monitoring information; a data analysis unit connected with the data processing and control unit for receiving the basic information and the pressure monitoring information with which the pressure regulation information is calculated and generated by the data analysis unit for transmissions of the pressure regulation information from the data analysis unit to the data processing and control unit, the data processing and transfer unit and the pressure control device sequentially.

Preferably, the data processing device further comprises a monitoring control unit connected with the data processing and control unit as well as the data processing and transfer unit for receiving the pressure monitoring information, and used to set a monitoring threshold for each air cushion units with which a warning signal is generated by the monitoring control unit and transmitted to outside environment by the data processing and transfer unit when anyone of the pressure monitoring information of the air cushion units exceeding the monitoring threshold.

Preferably, the data analysis unit further comprises an application analysis module recording the variation of the pressure monitoring information and collecting the same pressure range in the variation, and the same pressure range is labeled as a specific time interval for generation of service conditions of an air mattress in a distinct time interval.

The remote management, control and alert system further comprises a remote managing device connected and integrated wirelessly via a data transfer module to both the previously described integrated internal systems but more directly with the internal data processing device which relays system performance data such as pressure monitoring and pressure regulation information. It is also integrated and connected wirelessly with the pressure control device which allows the user to remotely control all pump functions. In addition, a location expression module captures the geographic location of the unit and presents this geographic location on a map display interface on the managing device. An information display module captures essential patient and user data, and presents the user data on an information input interface on the managing device.

The managing device further comprises a pump function control module which wirelessly connects and integrates with the internal pressure control device and provides one or a combination of tilt control mode, inflation/deflation cycle mode, normal care mode, therapy mode, sleep mode, panel lock mode, power switch and pressure or power alert and mute functions. When the monitoring unit detects a function fault then this is transmitted to the managing device for remote notification to carriers via the display or via text or email that there is a fault with the air mattress.

### BRIEF DESCRIPTION OF THE DRAWINGS

The descriptions of the present invention would be more understandable from the detailed description given herein and below and the accompanying figures which are provided for better illustration, and description. These figures are not limited for the present invention, and wherein:

FIG. 1 illustrates a relationship diagram between connected devices/units in the present invention.

FIG. 2 illustrates an integrated and connected system relationship diagram in the present invention.

FIG. 3 illustrates a relationship diagram of data transfer between the integrated devices/units in the present invention.

### DETAILED DESCRIPTION OF THE INVENTION

The following descriptions are embodiments about a remote management, control and alert system for a medical air alternation mattress; however they are not intended to limit the scope of the present disclosure.

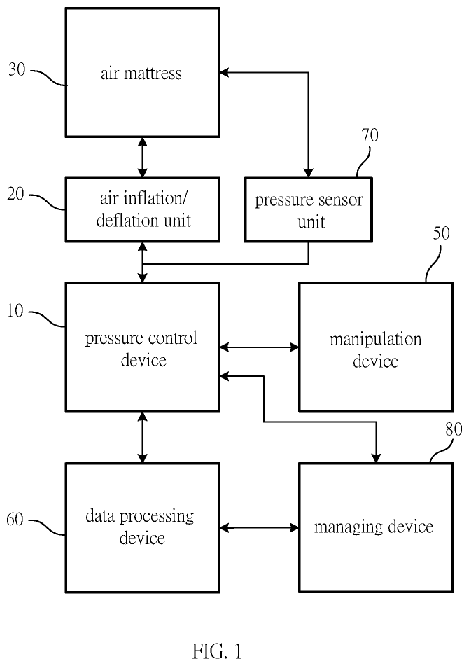

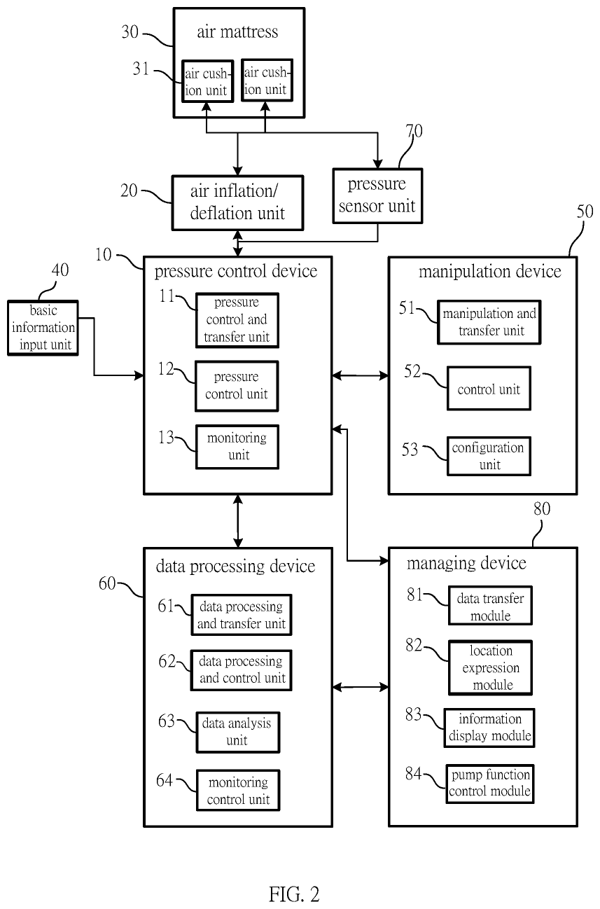

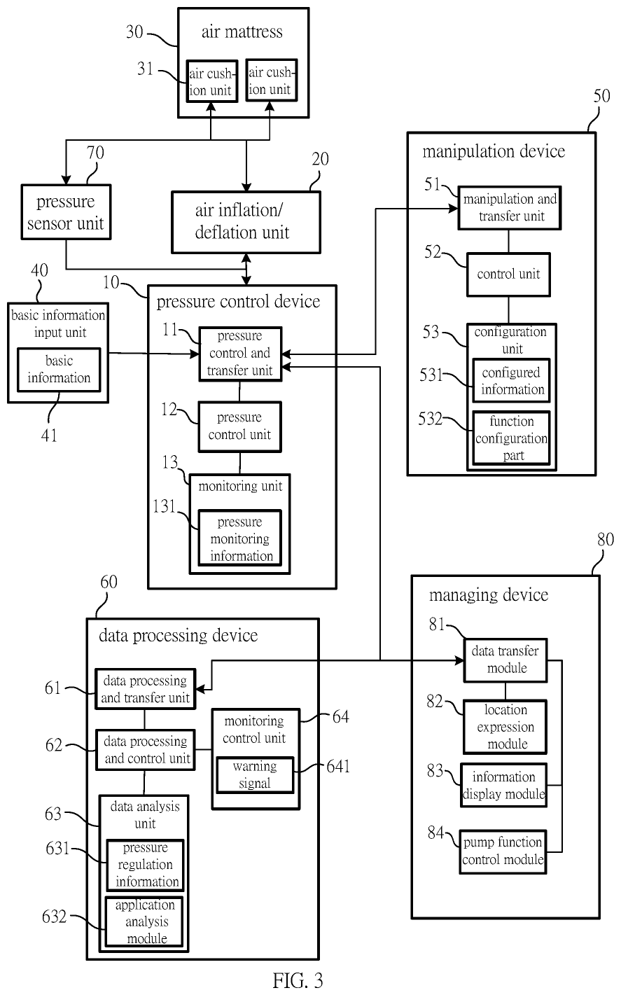

Referring to FIG. 1 to FIG. 3, which illustrates a relationship diagram between connected devices/units, an integrated and connected system relationship diagram, and a relationships diagram of data transfer between the integrated devices/units in the present invention, respectively. As shown in FIG. 1 to FIG. 3, a remote management, control and alert system for pressure regulation provided hereinafter comprises a pressure control device 10, which is connected with at least one air inflation/deflation unit 20 that connecting with a plurality of air cushion units 31 inside an air mattress 30 wherein the pressure control device 10 relies on the air inflation/deflation unit 20 to regulate pressures of the air cushion units 31 for legs, waist, head, etc. inside the air mattress 30. To inflate or deflate the air mattress 30 with the air inflation/deflation unit 20 controllably, the pressure control device 10 comprises a pressure control and transfer unit 11 connected with a basic information input unit 40 and a pressure control unit 12 for transmitting or receiving signals and/or information; an operator (assuming a nurse) enters basic information 41 of a user (assuming a patient) at the basic information input unit 40 which is designed as an independent component or embedded in the pressure control device 10 for sending the basic information 41 to the pressure control and transfer unit 11 and further outside environment. The pressure control unit 12 is connected with the air inflation/deflation unit 20 and at least one pressure sensor unit 70, which is installed on each air cushion unit 31 to detect real-time pressure of each air cushion unit 31, controls the air inflation/deflation unit 20, and receives real-time pressures of all air cushion units 31 detected by the pressure sensor unit 70. Moreover, the pressure control device 10 comprises a monitoring unit 13 connected with the pressure control unit 12 to which real-time pressures of all air cushion units 31 detected by the pressure sensor unit 70 is transmitted; the pressure control unit 12 sends real-time pressures of all air cushion units 31 to the monitoring unit 13 for generation of pressure monitoring information 131 based on the real-time pressures and transmission of pressure monitoring information 131 to the pressure control unit 12 and further the pressure control and transfer unit 11 from which the pressure monitoring information 131 is transmitted to outside environment.

The remote management, control and alert system for pressure regulation further comprises a manipulation device 50 connected with the pressure control device 10 to inflate or deflate the air mattress 30 and regulate pressures of all air cushion units 31 through the air inflation/deflation unit 20 connecting the pressure control unit 12 controllably. The manipulation device 50 comprises: a manipulation and transfer unit 51 connected with the pressure control and transfer unit 11 through which information is exchanged or transmitted between the manipulation and transfer unit 51 and the pressure control device 10; a control unit 52 connected with the manipulation and transfer unit 51; a configuration unit 53 connected with the control unit 52, generating configured information 531 based on a user's configurations and transmitting the configured information 531 to the control unit 52 from which the configured information 531 is forwarded to the pressure control device 10 through the manipulation and transfer unit 51 such that the pressure control device 10 relies on the configured information 531 to inflate or deflate one, some or all air cushion units 31 by controlling the air inflation/deflation unit 20.

In the manipulation device 50, the configuration unit 53 comprises a plurality of function configuration parts 532 by which a network connectivity and a combination of any two of a panel lock mode, a tilt control mode, a normal care mode and a therapy mode are created to enable inflation or deflation of an air cushion unit 31 for a specific body part, a panel lock function or a network connectivity. In the tilt control mode configured by a user, the pressure control device 10 controls the air inflation/deflation unit 20 to inflate the air cushion units 31 at the waist, shoulders and the head or deflate the air cushion units 31 at buttocks and legs optionally based on pressure status of these air cushion units 31 for an adequate tilt condition of the air mattress 10 on which a user lies down; in the therapy mode for a bed-ridden patient who fails to move his/her body freely, the air inflation/deflation unit 20 enables the inflation/deflation cycle at specific air cushion units 31 to turn over a user's body effectively for a therapy effect; the manipulation device 50 is latched or unlatched through the panel lock mode.

The remote management, control and alert system for pressure regulation further comprises a data processing device 60, for example, a cloud server, which is connected with at least one pressure control device 10 and further multiple pressure control devices 10 to receive information or signals from all pressure control devices 10 or send information or signals to all pressure control devices 10. The data processing device 60 comprises a data processing and transfer unit 61, a data processing and control unit 62 and a data analysis unit 63: the data processing and transfer unit 61 and the pressure control and transfer unit 11 are connected with each other to receive the basic information 41 and the pressure monitoring information 131; the data processing and control unit 62 and the data processing and transfer unit 61 are connected with each other to receive the basic information 41 and the pressure monitoring information 131. When the basic information 41 and the pressure monitoring information 131 are received by the data processing device 60 from the pressure control device 10 (for example, one pressure control device 10) and transmitted to the data analysis unit 63 via the data processing and transfer unit 61 and the data processing and control unit 62, pressure regulation information 631 is calculated and created by the data analysis unit 63 based on the pressure monitoring information 131; then, the pressure regulation information 631 is transmitted to the data processing and control unit 62 and the data processing and transfer unit 61 by the data analysis unit 63 and finally the pressure control device 10 through the data processing and transfer unit 61. Accordingly, the pressure control device 10 controls the air inflation/deflation unit 20 through which one, some, or all air cushion units 31 are inflated or deflated according to the pressure regulation information 631.

Furthermore, the data analysis unit 62 is equipped with an application analysis module 632 records the variation of the pressure monitoring information 131, and collects the same pressure range in the variation, and the same pressure range labeled as a specific time interval for analyses of service conditions of the air mattress 30. For example, when the pressure in each air cushion unit 31, which is compressed by an external force (resumed by a resilience) attributed to a user lying down on (getting up from) the air mattress 30, increases (decreases), a specific time interval that the user lying down on (getting up from) the air mattress 30 is labeled by the application analyses module 632 for statistic analysis of service conditions of the air mattress 30 at a distinct time interval.

The data analysis unit 62 further comprises a monitoring control unit 64 which is connected with the data processing and control unit 62 to receive the pressure monitoring information 131 and monitor pressures of each air cushion unit 31 from the pressure monitoring information 131. When pressure status of each air cushion unit 31 is unusual (due to leakages of the air cushion unit 31), a warning signal 641 is generated by the monitoring control unit 64 and displayed on a management interface. Moreover, the monitoring control unit 64 is connected with the manipulation and transfer unit 51 and the control unit 52 through the data processing and control unit 62 as well as the data processing and transfer unit 61 to access the manipulation device 50 by wired or wireless control.

Furthermore, the remote management, control and alert system for pressure regulation further comprises a managing device 80 connected with the data processing device 60 and comprising a data transfer module 81, a location expression module 82, an information display module 83 and a pump function control module 84. The managing device 80 is connected with the data processing and transfer unit 61 through the data transfer module 81 to receive the basic information 41 and equipped with the location expression module 81 and the information display module 82: the location expression module 82 captures the geographic location of the unit and presents this geographic location on a map display interface on the managing device 80; the information display module 83 captures essential patient and user data from the basic information 41 and presents the user data on an information input interface. The pump function control module 84 provides one or a combination of tilt control mode, inflation/deflation cycle mode, normal care mode, therapy mode, sleep mode, panel lock mode, power switch and pressure or power alert and mute functions for regulation of the air mattress 30, When the monitoring unit 13 detects a function fault then this is transmitted to the managing device 80 for remote notification to carriers via the display or via text or email that there is a fault with the air mattress 30. Wherein the tilt control mode, the normal care mode, the therapy mode and the panel lock mode are identical to functions of the plurality of function configuration parts 532.

Accordingly, the pressure status of each air cushion unit 31 in the air mattress 30 is regulated by the manipulation device 50 or the managing device 80 and processed and analyzed by the data processing device 60 for controllable pressures of all air cushion units 31 in the air mattress 30 by the pressure control device 10, a comfortable mattress on which a user lies down, and the air cushion units 31 of the air mattress 30 inflated or deflated by the air inflation/deflation unit 20.

The above descriptions explain the preferred embodiment in the present disclosure; however, the embodiment is not intended to limit the scope of the patent application. Any equivalent modification or change without departing from the spirit of the patent specification should be incorporated in the claims thereinafter.

What is claimed is:

1. A remote management, control and alert system for a medical air alternation mattress, comprising:
an air mattress with a plurality of air cushion units, each of the air cushion units connected with an air inflation/deflation unit to inflate/deflate independently, and each of the air cushion units equipped with a pressure sensor unit for generation of current pressure in each of the air cushion units;
an air supply device connected with the air inflation/deflation units respectively for supplying air to all of the air cushion units from the air supply device through each of the air inflation/deflation units;
a pressure control device connected with each of the air inflation/deflation units and all of the pressure sensor units, the pressure control device used to regulate actions of the air inflation/deflation unit, and adjusting pressures of the air cushion units for generation of pressure monitoring information based on pressures of the air cushion units, and transmitting the pressure monitoring information as well as a basic information set in the pressure control device to outside environment; and
a data processing device connected with the pressure control device, the data processing device used to receive the basic information and the pressure monitoring information for generation of pressure regulation information based on the pressure monitoring information, wherein the pressure control device relying on the pressure regulation information to regulate pressures of the air inflation/deflation unit.

2. The remote management, control and alert system for a medical air alternation mattress as claimed in claim 1 wherein the pressure control device comprises:
a pressure control and transfer unit connected with a configuration unit for receiving the basic information set at the configuration unit by a user and the current pressure of an air cushion unit detected by a pressure sensor unit, and transmitting the basic information to outside environment from the pressure control and transfer unit;
a pressure control unit connected with the pressure control and transfer unit for receiving current pressures of the air cushion units detected by the pressure sensor units and also connected with the air inflation/deflation unit for inflating/deflating the air mattress by the air inflation/deflation unit controllably; and
a monitoring unit connected with the pressure control unit through current pressures of the air cushion units are transmitted to the monitoring unit for generation of pressure monitoring information by the monitoring unit based on pressures of the air cushion units, and transmitting the pressure monitoring information from the monitoring unit to the pressure control unit, the pressure control and transfer unit, and outside environment sequentially.

3. The remote management, control and alert system for a medical air alternation mattress as claimed in claim 1 wherein the remote management, control and alert system further comprises a manipulation device connected with the pressure control device through which the air inflation/deflation unit connecting a pressure control unit is controlled for a regulated pressure of each of the air cushion units.

4. The remote management, control and alert system for a medical air alternation mattress as claimed in claim 3 wherein the manipulation device comprises:
a manipulation and transfer unit connecting and transmitting a configured information to the pressure control and transfer unit;
a configuration unit from the configured information based on a user's configurations is generated; and
a control unit connected with the configuration unit for receiving the configured information, and also connected with the manipulation and transfer unit for transmitting the configured information to the manipulation and transfer unit, the air inflation/deflation unit is regulated by the pressure control device controllably according to the configured information.

5. The remote management, control and alert system for a medical air alternation mattress as claimed in claim 4 wherein the configuration unit comprises:
a plurality of function configuration parts, each of the plurality of function configuration parts operated for generation of configured information;
at least one status display part installed around a function configuration part for displaying current configurations of the air mattress.

6. The remote management, control and alert system for a medical air alternation mattress as claimed in claim 4 wherein the data processing device comprises:
a data processing and transfer unit connected with the pressure control and transfer unit for receiving the basic information and the pressure monitoring information;
a data processing and control unit connected with the data processing and transfer unit for receiving the basic information and the pressure monitoring information; and
a data analysis unit connected with the data processing and control unit for receiving the basic information and the pressure monitoring information with which the pressure regulation information is calculated and generated by the data analysis unit for transmissions of the pressure regulation information from the data analysis unit to the data processing and control unit, the data processing and transfer unit and the pressure control device sequentially.

7. The remote management, control and alert system for a medical air alternation mattress as claimed in claim 6 wherein the data processing device further comprises a monitoring control unit connected with the data processing and control unit as well as the data processing and transfer unit for receiving the pressure monitoring information, and the monitoring control unit used to set a monitoring threshold for each of the air cushion units, the monitoring control unit generating a warning signal when anyone of the pressure monitoring information of the air cushion units exceeding the monitoring threshold, and transmitted the warning signal to outside environment by the data processing and transfer unit.

8. The remote management, control and alert system for a medical air alternation mattress as claimed in claim 6 wherein the data analysis unit further comprises an application analysis module recording the variation of the pressure monitoring information, and collecting the same pressure range in the variation, and the same pressure range labeled as a specific time interval for generation of service conditions of an air mattress in a distinct time interval.

9. A remote management, control and alert system for a medical air alternation mattress, comprising:
an air mattress with a plurality of air cushion units, each of the plurality of air cushion units is connected with an air inflation/deflation unit, inflated/deflated independently and equipped with a pressure sensor unit for generation of current pressure in each of the air cushion units;
an air supply device connected with the air inflation/deflation units respectively for supplying air to all of the air cushion units from the air supply device through each of the air inflation/deflation units;
a pressure control device connected with each of the air inflation/deflation units and all of the pressure sensor units, the pressure control device used to regulate actions of the air inflation/deflation unit, and adjusting pressures of the air cushion units for generation of pressure monitoring information based on pressures of the air cushion units, and transmitting the pressure monitoring information as well as a basic information set in the pressure control device to outside environment;
a data processing device connected with the pressure control device, the data processing device used to receive the basic information and the pressure monitoring information for generation of pressure regulation information based on the pressure monitoring information, wherein the pressure control device relying on the pressure regulation information to regulate pressures of the air inflation/deflation unit; and
a managing device connected with the data processing device and comprising:
a data transfer module receiving the basic information, the pressure monitoring information, and the pressure regulation information;
a location expression module capturing geographic locations from the basic information for presenting the geographic locations on a map display interface of the managing device; and
an information display module capturing an user data from the basic information for presenting the user data on an information input interface of the managing device.

10. The remote management, control and alert system for a medical air alternation mattress as claimed in claim 9 wherein the data processing device comprises:
a data processing and transfer unit connected with the pressure control and transfer unit for receiving the basic information and the pressure monitoring information;
a data processing and control unit connected with the data processing and transfer unit for receiving the basic information and the pressure monitoring information; and
a data analysis unit connected with the data processing and control unit for receiving the basic information and the pressure monitoring information, the pressure regulation information calculated and generated by the data analysis unit for transmitting the pressure regulation information from the data analysis unit to the data processing and control unit, the data processing and transfer unit, and the pressure control device sequentially.

11. The remote management, control and alert system for a medical air alternation mattress as claimed in claim 10 wherein the data processing device further comprises a monitoring control unit connected with the data processing and control unit as well as the data processing and transfer unit for receiving the pressure monitoring information, the monitoring control unit used to set a monitoring threshold for each of the air cushion units, the monitoring control unit generating a warning signal when anyone of the pressure monitoring information of the air cushion units exceeding the monitoring threshold, and transmitted the warning signal to outside environment by the data processing and transfer unit.

12. The remote management, control and alert system for a medical air alternation mattress as claimed in claim 10 wherein the data analysis unit further comprises an application analysis module recording the variation of the pressure monitoring information, and collecting the same pressure range in the variation, and the same pressure range labeled as a specific time interval for generation of service conditions of an air mattress in a distinct time interval.

13. The remote management, control and alert system for a medical air alternation mattress as claimed in claim 9 wherein the managing device further comprises a pump function control module which provides at least one or a combination of tilt control mode, inflation/deflation cycle mode, normal care mode, therapy mode, sleep mode, panel lock mode, power switch and pressure or power alert and mute functions for regulation of the air mattress.
